(19) United States
(12) Patent Application Publication
(10) Pub. No.: US 2007/0207470 A1
(43) Pub. Date: Sep. 6, 2007

(54) Humanization of antibodies using a putative ancient binary genetic code

(75) Inventors: Heinz Kohler, Lexington, KY (US); Raphael Finkel, Lexington, KY (US); Thomas Kieber-Emmons, Little Rock, AR (US)

Correspondence Address: JAMES H. MEADOWS AND MEDICUS ASSOCIATES, 2804 KENTUCKY, JOPLIN, MO, 64804, US

(73) Assignee: Chemclark, Inc.

(21) Appl. No.: 11/504,531

(22) Filed: Aug. 15, 2006

Related U.S. Application Data
(60) Provisional application No. 60/708,154, filed on Aug. 15, 2005.

(51) Int. Cl. C12Q 1/68; G06F 19/00; C07H 21/04; C12P 21/06; G01N 33/48; C12N 5/06; C07K 16/44

(52) U.S. Cl. 435/6; 435/69.1; 435/326; 435/320.1; 530/387.1; 536/23.53; 702/20

(57) ABSTRACT

A method for converting non-human antibodies into “humanized” versions having increased safety and efficacy employs a putative ancient binary genetic code to derive new antibody structures. The method entails determining the amino acid sequence of variable regions of heavy and/or light chains of the non-human antibody, identifying the framework sequences of the variable regions and conjoining them into a single heuristic sequence, which is then converted into a binary string equivalent. The binary string is searched in a binary version of a human protein database for a closest match to identify a reference sequence. The framework sequence of the non-human antibody is optionally modified to be identical with the reference sequence, then reassembled with complementarity-determining regions to produce a full-length heavy and/or light chain template. A DNA segment encoding the template is synthesized and expressed to afford the humanized version of the non-human antibody.

### REFERENCE TO RELATED APPLICATION

This application claims the benefit of U.S. Provisional Application 60/708,154, filed Aug. 15, 2005, the disclosure of which is incorporated herein by reference.

### FIELD OF THE INVENTION

The present invention relates to modification of nonhuman immunoglobulins to enhance their efficacy, specificity and safety.

### BACKGROUND OF THE INVENTION

Molecular biology over the last 10 years has elucidated the genome of humans, several animals, plants, bacteria, viruses and fungi. The genomic DNA sequences are deposited in a public database accessible for research and production of recombinant proteins. A major tool to access these data is the so-called NIH BLAST search that can detect genetic relationships in the genome of organisms. These searches are possible because the genetic code is universal for organisms, with some exceptions.

The universality of the genetic code led Crick in 1968 [Crick, F. (1968) J. Mol. Biol. 38: 367-379] to propose that the “allocation of codons to amino acids was entirely a matter of chance”. Since then, variations in codon usage have been discovered that suggest that the genetic code was not a “frozen accident” but has evolved over time [Knight, R. D. et al (1999) TIBS 24:241-247]. It is commonly believed that as life began, there were fewer amino acids than the current count of 20; a code less complex than the triplet code accommodates a smaller variety of amino acids. As has been previously proposed [Kohler, H. et al (2001) J. Mol. Rec., 14: 269-272] it is possible that primordial life operated with a binary genetic code consisting of two chemically different nucleotides, e.g., a purine and a pyrimidine. However, such a genetic code can only accommodate two different amino acids. This line of reasoning has led to the suggestion that early codon relationships exist between nucleotides and amino acids [Root-Bernstein, R. S. (1983), J. Theor. Biol. 100: 99-106].

The evolution of the genetic code has been reviewed recently [Knight, R. et al. (1999) TIBS, 24: 241-247]. It has been observed that a relation between codon usage and amino acid can be recognized in the nucleotide base occurring in the second position of the triplet code. In particular, the second base of the triplet code appears to sort the 20 amino acids into two hydropathy groups, i.e., purines A and G are associated with positive hydropathy, and pyrimidines C and U with negative hydropathy. Hydrophobic Tyr and Trp and hydrophilic Ser and Met are exceptions.

Further support for a binary code is found in the code ambiguity that occurs in primitive organisms which use variants of the standard code [Kohler, H. et al. (1995) J Mol. Recognition, 14: 269-272]. The fungus Candida uses the codon CUG to encode serine, whereas the standard code encodes leucine [Santos, M. et al. (1995) Nucleic. Acids Res. 23: 1481-1486]. This result suggests a hidden binary code in CUG, called “zero”, whereby serine and leucine derive from the same binary code. Similarly, Saccharomyces cerevisiae uses four of six codons, which in the standard code encode leucine, to encode threonine [Zimmer T. et al. (1995) Yeast, 11: 33-41]. Again, leucine and threonine are imputed to share the same binary code of “zero”.

Still further support for a primordial binary code comes from a sharing of the hidden binary code by different amino acids that control the folding of proteins. Analysis of the helix kinks in transmembrane proteins [Yohannan S., et al. (2004) Proc. Natl Acad. Sci USA 101: 959-963] shows that, in addition to proline in the position of bending, alanine, valine, phenylalanine and threonine are present. This set of amino acids carries the binary code “zero”. Evidently, important structural functions in protein folding preserve a primordial genetic code and are remnants of a primordial binary code.

Recently, an artificially ambiguous genetic code has been made [Pezo V. et al (2004) Proc. Natl Acad. Sci USA 101: 8593-8597] that confers growth advantage. The aminoacyl-tRNA synthetase for isoleucine has been mutated to allow both isoleucine and valine to use the same code triplet. Thus, a laboratory experiment has re-created an evolutionary ancient ambiguous code that uses an identical binary (zero) code word for isoleucine and valine.

Protein integration in the yeast genome has been compared with evolutionary distance to the protein's ortholog in C. elegans [Fraser, H. B. et al. (2002) Science 296: 750-752]. A correlation was detected between the evolutionary distance and the number of interactions showing the higher the network interactions were the greater the evolutionary distance. As the evolutionary rates of proteins and their networks are being discovered, it is increasingly apparent that protein-protein interacting surfaces are highly conserved in evolution. These observations imply that network building is closely linked to gene duplication and mutational specification suggesting that network building may have its origin at the onset of functional diversity. There is accumulating evidence that protein networks are an integral part of general protein evolution. Inasmuch as protein folding determines protein function, it also dictates protein contacts.

Translation of a protein sequence into a binary genetic code provides evidence for a primordial binary genetic code based on the hydropathy relationship of the second base in the coding nucleotide triplet. The binary code sequesters the amino acids into two categories, disregarding side chain differences in charge, size and hydropathy. Such a binary code better elucidates the evolution of proteins and their networks than the younger triplet code. In addition, the reduction of protein sequence data to a binary code set simplifies data management and analysis.

However, translation of the current genetic code back into the putative ancient binary code introduces errors, because of the ambiguity of codon usage for the amino acid serine. Four codons for serine have a pyrimidine, C, in the second base position, whereas the two other codons have a purine, G, in that position. Thus, in a reduction to the binary code from an amino acid sequence, the resulting binary sequence does not mirror the coding DNA sequence.

It has been noted that codon ambiguity can confer adaptive advantages in growth yield [Pezo V. et al., supra]. At the level of protein structure, similar ambiguities in amino acid have been documented. For example, analysis of the kinks in helical conformations can be achieved by a number of different amino acids, including the canonical proline, that all share the same binary code [Yohannan S., supra]. An example of amino acid ambiguity that does not produce variations in protein folds suggests a higher fidelity in binary code executed homology searches than achieved by searching at the level of a degenerate codon language, i.e., the current triplet code.

It is argued herein that searching for evolutionarily related genes with identical function, so-called ortholog genes, is more effective at the level of protein structure than at the level of the coding sequence, due to over-specification in the triplet code. Accordingly, in the present invention, a binary genetic sequence is extracted from a known amino acid sequence, because the function of a given gene is ultimately determined at the level of amino acids in the protein. This facilitates computerized homology searches. Moreover, it can enable the development of ortholog proteins that retain essential folding and hydropathic properties.

The principles of the present invention are applied to the problem of “humanization” of nonhuman antibodies. Typically, antibodies immunoreactive with a given antigen are initially obtained in the laboratory from nonhuman models. Great interest lies in “humanizing” such antibodies, i.e., modifying them so that they are both efficacious and tolerated by a human host. One approach proposes to find homologous human and nonhuman, typically murine, variable framework regions, on the theory that close chemical similarity in the framework regions is most important for providing toleration in the host while retaining affinity [See, e.g., Queen, C. et al, (1989) Proc. Natl Acad. Sci USA 86: 10029-10033 and U.S. Pat. Nos. 5,585,089, 6,180,370, and 7,022,500 (issued to Queen et al.)]. A second approach argues that similarly structured CDRs (complementarity-determining regions) will point to human frameworks that also support mouse CDRs with good retention of affinity [Hwang, W. et al. (2005) Methods, 36: 35-42]. Recently described is a laborious third approach that involves generation of a library of humanized heavy and light chain pairs from corresponding murine heavy and light chains. The humanized pairs are then screened for antigen binding [U.S. Pat. No. 7,087,409 (issued to Barbas, III et al.)]. Yet another proposal calls for ranking amino acid similarities between non-human CDRs and human CDRs obtained from a library of human antibody sequences, without need for comparing framework sequences [U.S. Pat. No. 6,881,557 (issued to Foote)].

We propose herein a new paradigm in which a simplified genetic code is employed to identify preexisting human variable region immuno-sequences having a high degree of homology with nonhuman sequences, with that information being used to generate minimally altered modifications of the nonhuman sequences.

### SUMMARY OF THE INVENTION

Based on insights obtained from our study of the putative primordial genetic code, binary strings can be constructed and employed to identify and generate humanized immunoglobulins. In this method, only the second base position of each triplet codon encoding a variable region amino acid of the nonhuman antibody is considered. A value of “1” is arbitrarily assigned to purine bases (A and G) appearing in the second position, which are imputed to encode amino acids having positive hydropathy; and the value “0” is assigned to pyrimidines (U and C), which encode amino acids having negative hydropathy. Human immunoglobulin gene databases can be screened with the summarizing nonhuman binary strings obtained previously to identify those nucleotide sequences having the greatest homology. Finally, whenever a mismatch occurs between the two strings, the amino acid at that position can be replaced with one having the correct hydropathic properties. The number of modifications necessitated by this method can be quite small. In addition, the inherent ambiguity in the encoding nucleotide and amino acid sequences affords great flexibility in the design of novel fusion proteins.

### BRIEF DESCRIPTION OF THE DRAWINGS

FIG. 1 depicts an algorithm according to the principles of the present invention. As shown, nonhuman antibody framework (FR) and CDR sequences are conjoined into searchable amino acid sequences. The conjoined FR sequence is searched against a human antibody database of known sequences.

FIG. 2 depicts an alternative algorithm of the invention, in which a conjoined nonhuman antibody CDR sequence is searched against a human antibody database of known CDR sequences in addition to searching against known FR sequences.

### DESCRIPTION OF THE INVENTION

Previously, a computerized search of a human genomic antibody database for orthologs to a known nonhuman antibody (NHA) would typically require a detailed knowledge of both of the human and nonhuman antibody genomes. This would require a determination of the pertinent genomic sequences of the antibody clone that secretes the NHA. To this end, the amino acid residue sequences of the heavy and/or light chain variable regions of the NHA would be determined by conventional methods, e.g., trypsin digest, N-terminal or C-terminal microsequencing, mass spectroscopy, and the like. The amino acid sequences thereby obtained would then be used to generate a random or reduced set of DNA oligonucleotides that would be used to probe the clone for hybridization. The genomic sequences bracketed by hybridizing probes would then be amplified by PCR, and the amplified DNA would be sequenced by conventional techniques to deduce the native codons encoding the NHA. The resulting nucleotide sequences would then be used to search for orthologs within the human database.

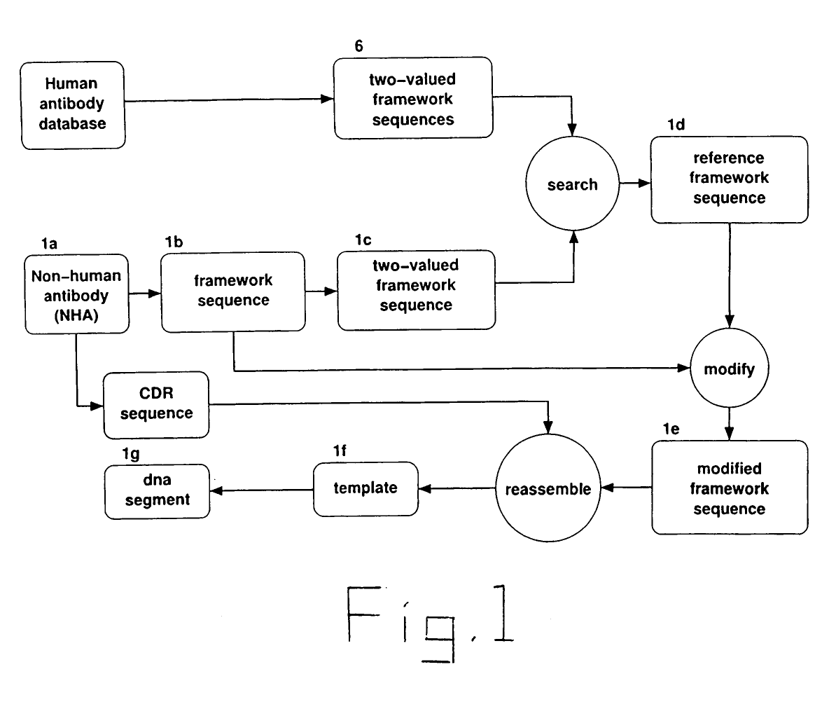

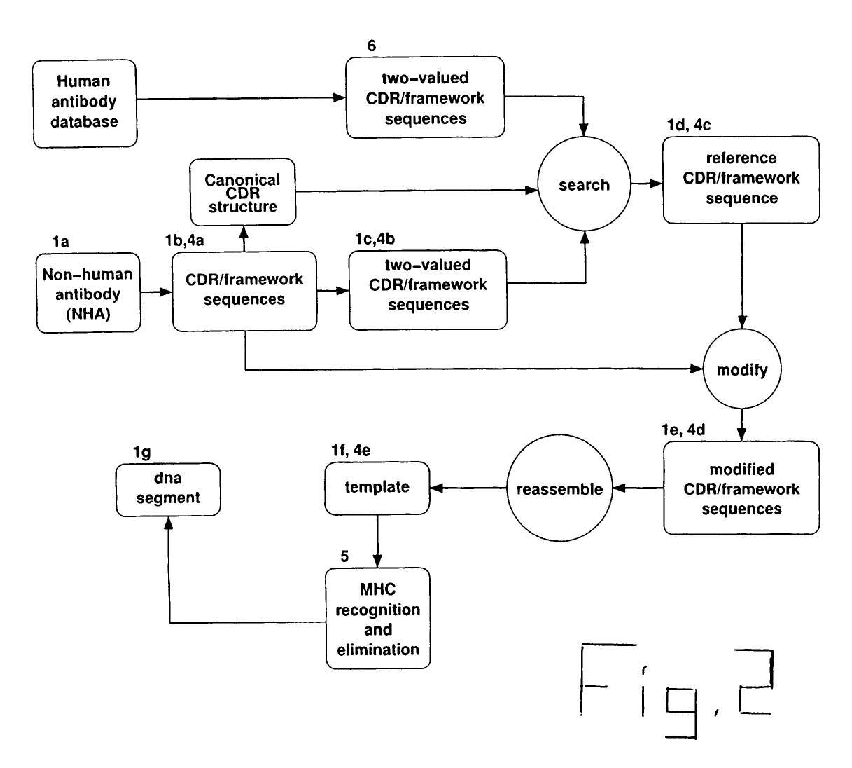

The present invention maintains that the previous approach over-specifies the problem and generates a frequently unnecessary level of detail. The present invention is simpler and avoids some of the more tedious recombinant DNA and analytical techniques outlined above. Algorithms for performing the present invention are illustrated with reference to FIGS. 1 and 2.

Referring to FIG. 1, the pertinent heavy and/or light chain variable regions of a NHA are determined by conventional means, vide supra. (1a) The framework regions (FRs) and/or complementarity-determining regions (CDRs) of the NHA are next identified with reference to known sequences and established protocols [E A Kabat, et al. MD, 1991] The FRs are conjoined into a single amino acid sequence, in order. (1b) Optionally, the CDRs are also conjoined, in order, into a single amino acid sequence, or the FRs and CDRs together are conjoined, in order, into a single amino acid sequence.

The single amino acid sequence obtained thereby is then converted into a two-valued (binary) string according to the primordial genetic code shown in Table 1. (1c)

TABLE 1
Putative Binary Primordial Genetic Code

| | | |
|---|---|---|
| Pyrimidines | C | Ser, Pro, Thr, Ala |
| (value = 0) | U | Phe, Leu, Ile, Met, Val |
| Purines | A | Tyr, His, Gln, Asn, Lys, Asp, Glu |
| (value = 1) | G | Cys, Trp, Arg, Gly |

As shown in Table 1, those amino acids encoded by a pyrimidine base in the second position of the standard triplet code are assigned the value “0”. These include Ser, Pro, Thr, Ala, Phe, Leu, Ile, Met and Val. Those amino acids “encoded” by a purine base in the second position are assigned the value “1”. These include Tyr, His, Gln, Asn, Lys, Asp, Glu, Cys, Trp, Arg, and Gly. (Strictly speaking, the codons for Ser are ambiguous since four are in the pyrimidine class and two are in the purine class; therefore, the predominating code is selected, which is the same as for Leu.)

The two-valued representation, sometimes referred to herein as a “probe”, is then used to screen, preferably by computer, a human antibody gene database that has likewise been converted to a two-valued representation [Kohler H. supra]. This process permits selection of those human framework sequences, sometimes referred to herein as “reference” sequences, showing the greatest degree of homology with the NHA framework sequences. (1d) A distance match algorithm looking for the sequence with minimal Hamming Distance [Dictionary of Algorithms and Data Structure, NIST, Black, P., ed.] from the probe is employed to discover the closeness of the match.

Based on the homology results obtained by the comparison of human and NHA binary strings, it may or may not be necessary to modify the NHA framework and/or CDR sequences to make it/them identical with the corresponding human framework and/or CDR sequences, thereby “humanizing” them. (1e) Typically, modification efforts are confined to replacing the amino acids in the non-identical positions of the NHA sequence(s) with the amino acids appearing in the corresponding positions of the reference sequence(s). Such modifications can be performed in situ by conventional recombinant DNA techniques, e.g., homologous recombination, point mutation with synthetic oligonucleotides, and the like, which alter the genome of the NHA-secreting clone and introduce the necessary changes. See, e.g., J. Sambrook, et al., Molecular Cloning: A Laboratory Manual, 3^{rd} ed., Cold Spring Harbor Laboratory Press, New York, 2001, and protocol updates available online from CSH Protocols Online.

Alternatively, expression vectors individually containing the nucleotide sequences that encode the respective FRs and/or CDRs of the NHA can be altered by conventional techniques, and followed by replication and excision with restriction enzymes to afford sufficient amounts of the desired DNA segments containing modifications. Direct PCR amplification of oligonucleotides encoding the NHA FRs and/or CDRs, employing primers containing the desired point mutation(s), can also be used to generate sufficient amounts of appropriately modified DNA segments. The desired humanized immunoglobulins can be expressed by the cell using standard transformation and culture techniques. See, e.g., Sambrook, vide supra.

DNA segments encoding the (un)modified NHA FRs and/or CDRs can be reassembled by standard ligation techniques to generate a complete antibody gene template. (1f) Alternatively, a full-length template can be synthesized directly, with or without modification, based on the information obtained above. The template can then be introduced into a cell capable of expressing the humanized antibody, optionally employing an expression vector that contains the template. (1g)

An alternative aspect of the invention is depicted in FIG. 2. In this aspect, binary strings representing both a contiguous framework (1c) and contiguous CDR sequences (4b) are constructed. Similarly, a binary database of known human antibody framework and CDR sequences (6) is searched for the closest matches with the NHA string(s). (1d,4c) Matching can be further refined by consideration of known canonical structures relating to the CDRs [Chothia, C., et al. (1987) J Mol Biol. (1987) 20:901-917], which information is used to accept or reject candidate structures. The minimal-distance results identify which, if any, modifications need to be made in the NHA FR and CDR sequences. As before, the FRs and CDRs are modified (1e,4d), as needed, and reassembled to generate a DNA template (1f, 4e). The template is then subjected to MHC recognition and elimination (5) to afford a final DNA segment (1g) that is introduced into cells capable of expressing the candidate “humanized” immunoglobulin.

The invention is further illustrated with reference to a specific example for humanizing the murine T15 antibody [BLAST, gi|346800]. The variable heavy chain FRs of the T15 antibody, when represented as a single, contiguous amino acid sequence (absent the CDRs), have the following formula:

(SEQ ID NO: 1)

EVKLVESGGGLVQPGGSLRLSCATSGFTFS/WVRQPPGKRLEWIA/YTTE
YSASVKGRFIVSRDTSQSILYLQMNALRAEDTAIYYCAR

In the formula, the two diagonals indicate the positions where different parts of the framework are conjoined. The three regions correspond to positions 1-30, 36-51, and 57-94 of the standard Kabat alignment of heavy chains. The amino acids in boldface indicate those residues differing from the closest-match human antibody, vide infra.

The single T15 FR sequence translates according to the code shown in Table 1 into the following binary string (where numbers in boldface correspond to the aforementioned amino acid residues):
101001011100101100100100010000101100111011001001100001110000110010001010 100101100011101

A computerized minimal-distance algorithm that searches a BLAST human genome database for closest matches to the T15 sequence identifies an amino acid sequence, designated 12-2′CL, having the following formula and binary translation:

(SEQ ID NO: 2)

EVqLVeSgggLVqPggSLrLScAASgFTFSwVrqAPgkgLewVgrTTeYA
ASVkgrFTISrddSknSLyLqMnSLkTedTAVyycAr

101001011100101100100100010000101100111011011001100001110000111011001010 100101100011101
   In this formula, capital letters signify amino acid residues encoded by a binary 0 and lower case letters signify residues encoded by a binary 1; serine is always binary 0. Positions 44, 63, and 66 differ in binary code and are indicated in boldface type.

Based on these results, it is desired to modify the T15 variable heavy chain FR sequences to incorporate the amino acid residues found in 12-2′CL:

(SEQ ID NO: 3)

EVKLVESGGGLVQPGGSLRLSCATSGFTFSWVRQPPGKRLEWIGYTTEYS
ASVKGRFIVSRDDSQNILYLQMNALRAEDTAIYYCAR

The present invention has been described with reference to certain examples for purposes of explanation and clarity of understanding. It should be appreciated by the skilled practitioner that obvious improvements and modifications can be practiced within the scope of the appended claims.

What is claimed is:

1. A method of converting a non-human antibody, which has potential diagnostic or therapeutic utility, into a humanized immunoglobulin comprising:
(a) determining at least one amino acid sequence of variable regions of heavy and/or light chains of the non-human antibody (NHA);
(b) identifying the framework sequences of the non-human antibody and conjoining the same into a single heuristic amino acid sequence, in framework order;
(c) converting the heuristic sequence into a probe, which probe is comprised of a plurality of binary values, with each value representing an amino acid in the heuristic sequence, wherein a purine appearing in the second base position of a triplet codon encoding an amino acid is assigned a first value, and a pyrimidine appearing in the second base position is assigned the second value, with serine being assigned the same value as leucine;
(d) providing a human antibody database comprised of two-valued strings, which strings represent conjoined framework sequences of human antibody amino acid sequences, and are assigned the same binary values as the probe;
(e) searching the strings of the database for a minimal-distance match with the probe, thereby identifying a reference sequence;
(f) optionally, modifying a framework sequence of the non-human antibody in at least one position where a value of the probe is not identical with the corresponding value of the reference sequence, by replacing the amino acid in such position with the amino acid in the corresponding position of the reference sequence;
(g) reassembling the framework sequences, optionally modified, of the non-human antibody with the corresponding complementarity-determining regions (CDRs) to produce a full-length heavy- and/or light-chain variable region template;
(h) synthesizing a DNA segment encoding the full-length template;
(i) introducing the DNA segment into a cell capable of expressing the humanized immunoglobulin; and
(j) expressing the humanized immunoglobulin encoded by the DNA segment.

2. The method of claim 1, wherein the pyrimidine is assigned the value 0 and the purine is assigned the value 1.

3. The method of claim 1, wherein the minimal-distance match is characterized by a two-valued sequence that differs from the probe sequence in the fewest number of positions.

4. The method of claim 1, wherein the searching is restricted to those strings in the database that have the same canonical structure as the probe.

5. The method of claim 1, further comprising identifying the CDR amino acid sequences of the NHA, conjoining the same into a single sequence in CDR order, and converting the single sequence into a two-valued representation.

6. The method of claim 5, wherein the two-valued representation is the same as for framework sequences.

7. The method of claim 5, further comprising providing a human antibody database comprising two-valued strings of conjoined CDR sequences.

8. The method of claim 7, comprising searching the human database for a minimal-distance match with the two-valued representation of the CDRs, which match serves as a reference sequence.

9. The method of claim 8, comprising modifying the CDRs of the NHA in those positions where the two-valued representation is not identical with the reference sequence, by replacing the amino acid in a non-identical position of the NHA with the amino acid appearing in the corresponding position of the reference sequence.

10. The method of claim 9, comprising reassembling the modified CDRs and modified frameworks of the NHA to produce a full-length heavy- and/or light-chain variable region template thereof.

11. The method of claim 10, further comprising modifying the template by identifying regions recognized by the human major histocompatibility complex and modifying the same to abolish such recognition.

12. A database comprising a two-valued representation of human antibody amino acid sequences.

13. The database of claim 12, wherein individual entries comprise the framework, CDRs, and canonical structures of the antibody.

14. The database of claim 12, wherein the amino acid sequences are at least 10 residues in length.

15. A method of generating the database of claim 12, wherein the framework sequences of a human antibody are conjoined into a single sequence, which single sequence is converted into a two-valued representation.

16. The method of claim 15, wherein the CDRs of a human antibody are conjoined into a single sequence, which single sequence is converted into a two-valued representation.


SEQUENCE LISTING (3 sequences)

SEQ ID NO: 1 - protein, 87 aa, mouse
  PEPTIDE (1)..(30): framework region FR1 of T15 antibody VH chain
  PEPTIDE (31)..(44): framework region FR2 of T15 antibody VH chain
  PEPTIDE (45)..(87): framework region FR3 of T15 antibody VH chain
EVKLVESGGGLVQPGGSLRLSCATSGFTFSWVRQPPGKRLEWIAYTTEYSASVKGRFIVSRDTSQSILYLQMNALRAEDTAIYYCAR

SEQ ID NO: 2 - protein, 87 aa, human
EVQLVESGGGLVQPGGSLRLSCAASGFTFSWVRQAPGKGLEWVGRTTEYAASVKGRFTISRDDSKNSLYLQMNSLKTEDTAVYYCAR

SEQ ID NO: 3 - protein, 87 aa, Artificial Sequence
  MISC_FEATURE (1)..(87): Description of artificial sequence Conjoined variable heavy chain framework regions of T15 murine antibody modified withhuman residues at positions 44, 63 and 66
EVKLVESGGGLVQPGGSLRLSCATSGFTFSWVRQPPGKRLEWIGYTTEYSASVKGRFIVSRDDSQNILYLQMNALRAEDTAIYYCAR